(19) United States
(12) Patent Application Publication
(10) Pub. No.: US 2024/0165344 A1
(43) Pub. Date: May 23, 2024

(54) Syringe Needle Shield with Integrated Disinfectant

(71) Applicant: Becton, Dickinson and Company, Franklin Lakes, NJ (US)

(72) Inventors: Shishir Prasad, Ramsey, NJ (US); Manish Kumar, Bengaluru (IN); Karthik MR, Bangalore (IN)

(21) Appl. No.: 17/992,438

(22) Filed: Nov. 22, 2022

(51) Int. Cl. A61M 5/32; A61M 5/00

(52) U.S. Cl. CPC A61M 5/3202; A61M 5/001

(57) ABSTRACT

A needle cover includes a needle shield, a mount coupled to the needle shield, and a disinfecting pad coupled to the mount. The disinfecting pad is fixedly coupled to the needle shield with an adhesive, and wherein the disinfecting pad is a porous material impregnated with a disinfectant. The disinfecting pad is a precut foam having a plurality of columnar structures.

### BACKGROUND OF THE DISCLOSURE

### Field of the Disclosure

The present disclosure relates generally to a syringe needle shield.

### Description of Related Art

The most common technique for disinfecting a patient's skin at an injection site is swabbing a disinfectant to the skin prior to a surgical or non-surgical procedures. An example of skin preparation includes wiping (or swabbing) with alcohol or other disinfectant solution before administering an injectable. The current technique has many drawbacks, including the inconvenience of having to carry the individually packaged alcohol swabs, as they take up additional space and are purchased and carried in bulk. Another drawback is that alcohol swabs are typically small and easy to misplace. Further, many diabetics who commonly have to administer injectables have dexterity problems, making it difficult to open and unfurl antiseptic swabs. Additionally, it may be inefficient because single-handed operation may not be possible when using alcohol wipes packaged separately, and when alcohol wipes are taken out of the package, some of the disinfectant remains in the package which is not used. Yet another drawback is this technique requires direct contact with alcohol wipe during disinfection, thus potentially contaminating the site.

Accordingly, those skilled in the art continue with research and development efforts in the field of disinfecting prior to administering an injectable.

### SUMMARY OF THE INVENTION

Disclosed is a needle cover.

In one example, the needle cover includes a needle shield and a disinfecting pad coupled to the needle shield.

The disinfecting pad may have a thickness of approximately 2 mm to approximately 4 mm. The disinfecting pad may be substantially round. The disinfecting pad may have a diameter of approximately 5 mm to approximately 15 mm. The disinfecting pad may be substantially rectangular. The disinfecting pad may have a width W approximately 10 mm to approximately 30 mm and a height of approximately 5 mm to approximately 15 mm. The disinfecting pad may be a porous material impregnated with a disinfectant. The disinfecting pad may be a precut foam having a plurality of columnar structures. The disinfecting pad may be a sponge. The disinfecting pad may be impregnated with a disinfectant, such as approximately 70% isopropyl alcohol or Chlorhexidine in an IPA-based disinfectant.

The needle cover may include a mount coupled to the needle shield. The disinfecting pad may be coupled to the mount with an adhesive. The mount may be removably coupleable to the needle shield. The mount may be integrated with the needle shield such that they are a single, monolithic structure. The mount may include a pad mount coupled to the disinfecting pad and a shield sleeve surrounding a distal end of the needle shield.

The needle cover may further include a protective film coupled with the disinfecting pad.

Also disclosed is a syringe.

In one example, the syringe includes a barrel a plunger at least partially received in the barrel a needle hub coupled to the barrel, a needle coupled to the needle hub, and a needle cover removably coupled to the needle hub. The needle cover includes a needle shield and a disinfecting pad coupled to the needle shield.

A proximal end of the needle cover may be removably coupled to the needle hub.

Also disclosed is a method for administering an injectable into an injection site with a syringe including a barrel, a needle cover coupled to the barrel having a needle shield, a disinfecting pad coupled to the needle shield, and a protective film coupled to the disinfecting pad.

In one example, the method includes removing the protective film. The method further includes swabbing the injection site with the disinfecting pad. The method further includes uncoupling the needle cover from the barrel. The method further includes administering the injectable into the injection site.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective schematic view of a needle cover;

FIG. 2 is a top schematic of a portion of the needle cover of FIG. 1;

FIG. 3 is a perspective schematic view of a needle cover;

FIG. 4 is a side schematic of a portion of the needle cover of FIG. 3;

FIG. 5 is a perspective schematic view of a needle cover;

FIG. 6 is a top schematic of a portion of the needle cover of FIG. 5;

FIG. 7 is a perspective schematic view of a portion of a needle cover;

FIG. 8 is a perspective schematic view of a portion of a needle cover;

FIG. 9 is a perspective schematic view of a syringe;

FIG. 10 is a perspective schematic view of a needle cover;

FIG. 11 is a perspective schematic view of a syringe;

FIG. 12 is cross-sectional side schematic of a portion of the syringe of FIG. 11; and

FIG. 13 is a flowchart of a method for administering an injectable into an injection site.

### DETAILED DESCRIPTION

Spatial or directional terms, such as “left”, “right”, “inner”, “outer”, “above”, “below”, and the like, are not to be considered as limiting as the disclosure can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

Reference herein to “one or more examples” means that one or more feature, structure, or characteristic described in connection with the example is included in at least one implementation. The phrase “one or more examples” in various places in the specification may or may not be referring to the same example.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

Although the disclosure has been described in detail for the purpose of illustration based on what is currently considered to be the most practical and preferred embodiments, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

The disclosure provides a streamlined means for disinfecting a patient's skin prior to administering an injectable. In one aspect, the disclosed is a needle shield assembly integrated with a disinfectant that is bonded to or integrated with the needle shield. Integrating a disinfecting device with a syringe enhances compliance of proper skin prep before injecting medication, which is oftentimes not achieved if proper disinfecting means are not provided at the patient's bedside. The disclosed design eliminates the need to look/search for disinfecting devices e.g., alcohol swabs, wipes, etc. but integrating the disinfectant with the needle shield assembly. After swabbing, the needle shield along with the disinfecting cap is removed and the injection is administered.

The disclosure may be useful for all types of medical procedures requiring needles, e.g., vaccination, nutrients, or anesthesia. This disclosure eliminates the inadvertent contamination and potential associated complications associated with alcohol wipes, which is currently widely used for skin disinfection. Alcohol wipes, by design, does not provide aseptic non-touch technique for disinfecting the skin.

The disclosure utilizes a disinfectant pad, such as a sponge, soaked in disinfectant is used to prepare and disinfect the injection site before administering medication. In one example, the disinfectant pad is a sponge or foam material integrated into the distal end of a needle shield, the disinfectant pad being impregnated with 70% IPA as disinfectant. The disclosed can be used to swab, in a similar manner to using a marker or applicator, once the alcohol sponge is exposed. This allows for an aseptic non-touch technique (ANTT).

In one aspect, the needle shield holds the IPA-soaked sponge in a cavity of the distal end of needle shield. The overall design helps users by providing an ergonomic solution for site preparation compared to conventional methods e.g., using wipes. The barrel or the needle shield of the syringe can serve as the handle to hold and provide better stability and usability to user for effective swabbing. The disclosure may include a top web peel or film that seals the soaked sponge from the external environment, thus ensuring container closure integrity.

The disclosed design is compatible with commercially-available needle shields, such as those used in hypodermic syringes. The disclosed design may function as an additional disinfecting device that is attachable to a commercially-available needle shield.

Referring generally to FIGS. 1-12, disclosed is a needle cover 100. The needle cover 100 is a disinfecting assembly to simplify the process of disinfecting an injection site on a patient prior to injecting medication via a needle of a syringe. The needle cover 100 includes a needle shield 110. The needle shield 100 may be any commercially available needle shield 100.

Referring specifically to FIG. 1, the needle cover 100 includes a mount 120 coupled to the needle shield 110. The mount 120 may be removably coupleable to the needle shield 110 such that it is configured to slide and/or snap on and off of an existing needle shield 110. In another example, the mount 120 may be integrated with the needle shield 110 such that they are a single, monolithic structure. The mount 120 may include a pad mount 122 coupled to the disinfecting pad 130. The pad mount 122 may be of any size or shape to mirror the size and shape of the disinfecting pad 130 described below. For example, the pad mount 122 may be substantially round, oval, rectangular, square, triangular, or any shape matching that of the disinfecting pad 120. The mount 120 may further include a shield sleeve 124 coupled to a distal end 112 of the needle shield 110. In one example, the shield sleeve 124 surrounds a distal end 112 of the needle shield 110 such that it nests over the distal end 112 of the needle shield 110, forming a cap. In one example, the shield sleeve 124 snaps or clicks onto the distal end 112 of the needle shield 110. In another example, the shield sleeve 124 slides over the distal end 112 of the needle shield 110.

Referring to FIGS. 1-12, the needle cover 100 includes a disinfecting pad 130 coupled to the mount 120. The disinfecting pad 130 may be coupled to the mount 120 with an adhesive, such as a glue. The adhesive may be thermally cured to seal the disinfecting pad 130 to the mount 120 and/or to the needle shield 110. The disinfecting pad 130 may be a porous material impregnated with a disinfectant. In one example, the disinfecting pad 130 is impregnated with an alcohol. In one specific example, the disinfecting pad 130 is impregnated with a disinfectant, such as approximately 70% isopropyl alcohol (IPA) or Chlorhexidine in an IPA-based disinfectant.

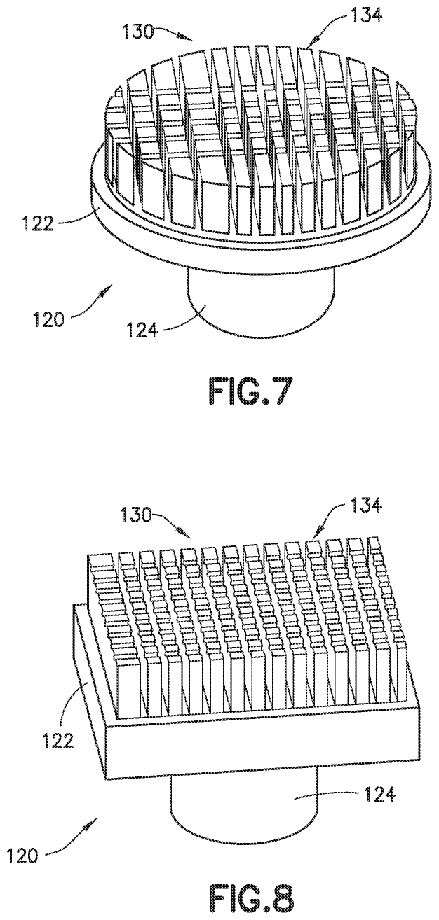

In one example, the disinfecting pad 130 is a sponge. In another example, the disinfecting pad 130 is a foam material. In yet another example, the disinfecting pad 130 is a precut foam having a plurality of columnar structures 134, see FIGS. 7 and 8. The plurality of columnar structures 134 may advantageously provide additional surface are for holding disinfectant, actively scrubbing the site, and disinfecting a patient prior to receiving a medicament injection.

The disinfecting pad 130 may be of any size or shape needed for adequately holding enough disinfectant to thoroughly disinfect a patient's injection site prior to receiving an injection. In one example, the disinfecting pad 130 has a thickness T of approximately 2 mm to approximately 4 mm, and more specifically approximately 3.5 mm, see FIG. 4. In one particular configuration, the disinfecting pad 130 may have a volume upon compression (against the prep site) of at least about 0.476 cc, as determined by a dimension of 28 mm×34 mm×0.5 mm (thick)=476 mm3=0.476 cc. The size of the disinfecting pad 130 is designed in such a way that the volume of IPA that it can hold based on saturation limit is at least equal to the volume of IPA filled in existing Alcohol wipes/pads to attain the needed disinfection.

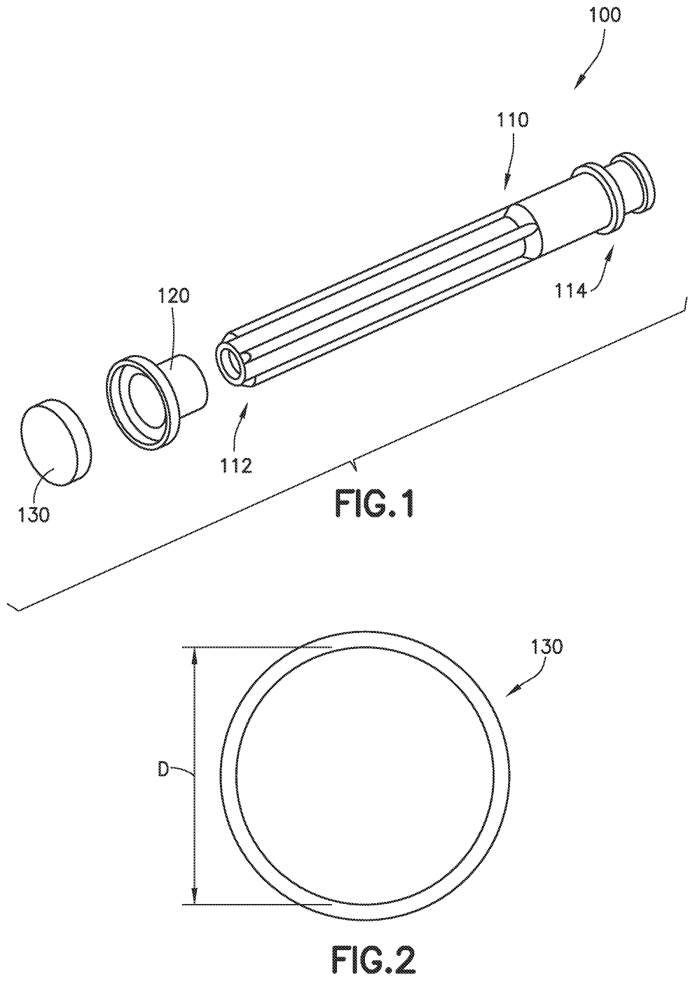

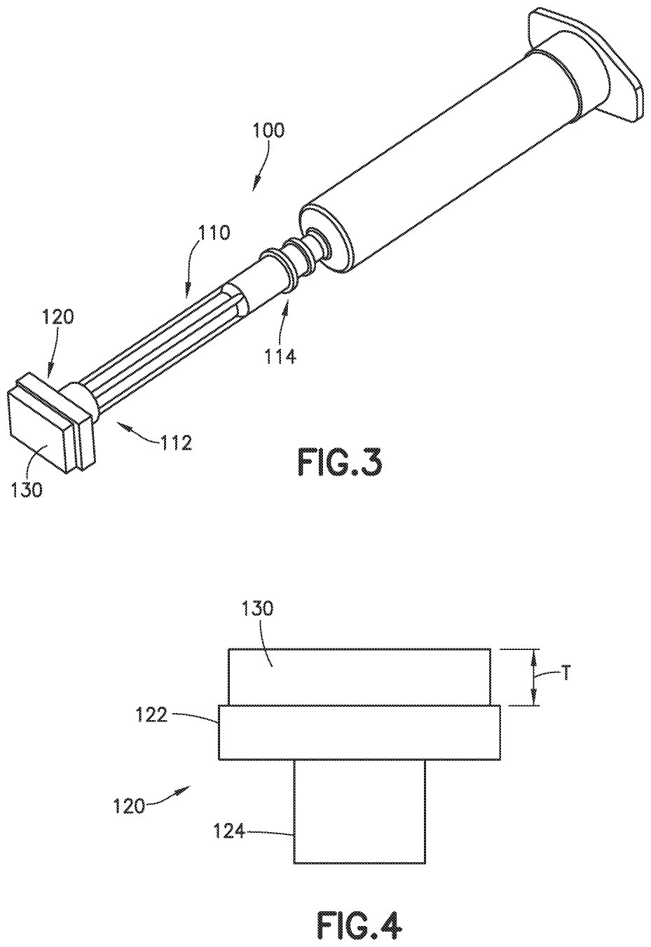

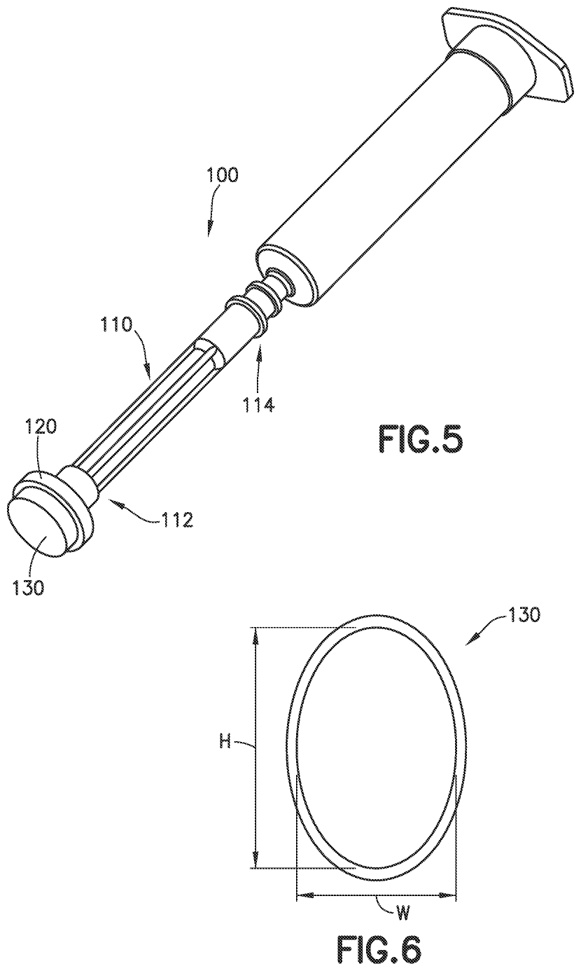

Referring to FIGS. 1 and 2, the disinfecting pad 130 may be substantially round and have a diameter D of approximately 5 mm to approximately 15 mm. In another example, the disinfecting pad 130 may be substantially rectangular, see FIGS. 3 and 4. In the example of having a substantially rectangular shape, the disinfecting pad 130 may have a width W approximately 10 mm to approximately 30 mm and a height H of approximately 5 mm to approximately 15 mm. Referring to FIGS. 5 and 6, the disinfecting pad 130 may be substantially oval shaped.

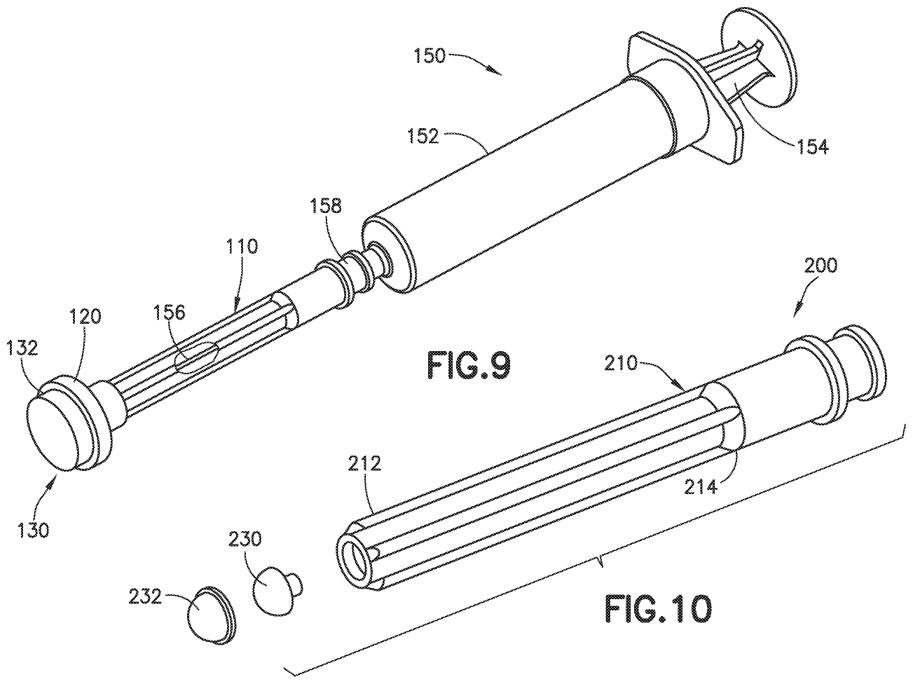

Referring to FIG. 9, the needle cover 100 may further include a protective film 132 coupled with the disinfecting pad 130. The protective film 132 may be peelable from the disinfecting pad 130. The protective film 132 may serve as a barrier to keep the disinfectant in the disinfecting pad 130 and to keep the disinfecting pad 130 from becoming contaminated prior to use. The protective film 132 may cover the entire exposed portion of the disinfecting pad 130, and may further extend beyond the disinfecting pad 130 to at least a portion of the mount 120 to ensure the disinfecting pad 130 is completely protected from contamination.

Referring to FIG. 9, disclosed is a syringe 150. The syringe 150 includes a barrel 152, a plunger 154 at least partially received in the barrel 152, a needle hub 158 coupled to the barrel 152, and a needle 156 coupled to the needle hub 158. In one example, a proximal end 114 of the needle cover 100 is removably coupled to the needle hub 158.

Still referring to FIG. 9, the syringe 150 further includes a needle cover 100 removably coupled to the needle hub 158. The needle cover 100 includes a needle shield 110, a mount 120 coupled to the needle shield 110, and a disinfecting pad 130 coupled to the mount 120 as shown and described herein. In one example, the disinfecting pad 130 is impregnated with an alcohol, such as approximately 70% isopropyl alcohol or chlorohexidine in an IPA-based disinfectant.

Referring to FIGS. 9-12, disclosed is another example of a needle cover 200. In one example, the needle cover 200 includes a needle shield 210. The needle shield 200 may be any commercially available needle shield 200 or may be specifically designed to receive a disinfecting pad 230.

Still referring to FIGS. 9-12, the needle cover 200 includes a disinfecting pad 230 coupled directly to a distal end 212 of the needle shield 210. The disinfecting pad 230 may be coupled to and nest with the distal end 212 of the needle shield 210 with an adhesive, such as a glue. The adhesive may be thermally cured to seal the disinfecting pad 230 to the needle shield 210. The disinfecting pad 230 may be a porous material impregnated with a disinfectant. In one example, the disinfecting pad 230 is impregnated with an alcohol. In one specific example, the disinfecting pad 230 is impregnated with approximately 70% isopropyl alcohol.

In one example, the disinfecting pad 230 is a sponge. In another example, the disinfecting pad 230 is a foam material. In yet another example, the disinfecting pad 230 is a precut foam having a plurality of columnar structures 234. The plurality of columnar structures 234 may advantageously provide additional surface are for holding disinfectant actively scrubbing the site, and disinfecting a patient prior to receiving a medicament injection.

The disinfecting pad 230 may be of any size or shape needed for adequately holding enough disinfectant to thoroughly disinfect a patient's injection site prior to receiving an injection. In one example, the disinfecting pad 230 has a thickness T of approximately 2 mm to approximately 4 mm. The disinfecting pad 230 may have a volume upon compression (against the prep site) of at least about 0.476 cc, as determined by a dimension of 28 mm×34 mm×0.5 mm (thick)=476 mm3=0.476 cc. The size of the disinfecting pad 130 is designed in such a way that the volume of IPA that it can hold based on saturation limit is at least equal to the volume of IPA filled in existing Alcohol wipes/pads to attain the needed disinfection. The disinfecting pad 230 may be substantially round and have a diameter D of approximately 5 mm to approximately 15 mm. In another example, the disinfecting pad 230 may be substantially rectangular. In the example of having a substantially rectangular shape, the disinfecting pad 230 may have a width W approximately 10 mm to approximately 30 mm and a height H of approximately 5 mm to approximately 15 mm.

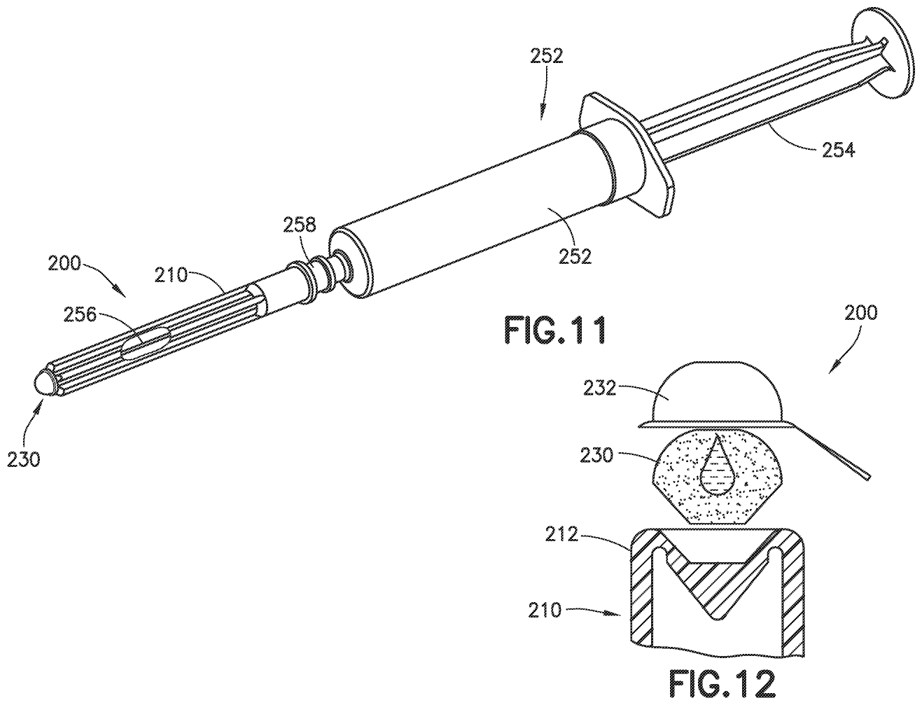

Referring to FIGS. 11 and 12, the needle cover 200 may further include a protective film 232 coupled with the disinfecting pad 230. The protective film 232 may be peelable from the disinfecting pad 230. The protective film 232 may serve as a barrier to keep the disinfectant in the disinfecting pad 230 and to keep the disinfecting pad 230 from becoming contaminated prior to use. The protective film 232 may cover the entire exposed portion of the disinfecting pad 230, and may further extend beyond the disinfecting pad 230 to at least a portion of the mount 220 to ensure the disinfecting pad 230 is completely protected from contamination.

Referring to FIG. 12, disclosed is a syringe 250. The syringe 250 includes a barrel 252, a plunger 254 at least partially received in the barrel 252, a needle hub 258 coupled to the barrel 252, and a needle 256 coupled to the needle hub 258. In one example, a proximal end 214 of the needle cover 200 is removably coupled to the needle hub 258.

Still referring to FIGS. 11 and 12, the syringe 250 further includes a needle cover 200 removably coupled to the needle hub 258. The needle cover 200 includes a needle shield 210, a mount 220 coupled to the needle shield 210, and a disinfecting pad 230 coupled to the mount 220 as shown and described herein. In one example, the disinfecting pad 230 is impregnated with an alcohol, such as approximately 70% isopropyl alcohol or chlorohexidine in an IPA-based disinfectant.

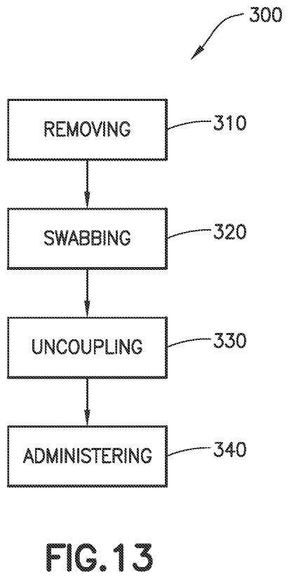

Referring to FIG. 13, disclosed is a method 300 for administering an injectable into an injection site with a syringe 150 comprising a barrel 152, a needle cover 100 coupled to the barrel 152 having a needle shield 110, a mount 120 coupled to the needle shield 110, a disinfecting pad 130 coupled to the mount 120, and a protective film 132 coupled with the disinfecting pad 130. The method 300 may be performed with the needle cover 100 and syringe 150 as shown and described herein.

In one example, the method 300 includes removing 310 the protective film 132. The method 300 further includes swabbing 320 the injection site with the disinfecting pad 130. The method 300 further includes uncoupling 330 the needle cover 100 from the barrel 152. The method 300 further includes administering 340 the injectable into the injection site.

Although non-limiting embodiments have been described in detail for the purpose of illustration and description, it is to be understood that such detail is solely for that purpose and that embodiments are not limited to the disclosed embodiments, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. A needle cover comprising:
a needle shield; and
a disinfecting pad coupled to the needle shield, wherein the disinfecting pad is fixedly coupled to the needle shield with an adhesive, and wherein the disinfecting pad is a porous material impregnated with a disinfectant.

2. The needle cover of claim 1, wherein the disinfecting pad is a precut foam having a plurality of columnar structures.

3. The needle cover of claim 1, wherein the disinfecting pad has a thickness (T) of approximately 2 mm to approximately 4 mm.

4. The needle cover of claim 1, wherein the disinfecting pad is substantially round.

5. The needle cover of claim 4, wherein the disinfecting pad has a diameter (D) of approximately 5 mm to approximately 15 mm.

6. The needle cover of claim 1, wherein the disinfecting pad is substantially rectangular.

7. The needle cover of claim 1, wherein the disinfecting pad has a width (W) approximately 10 mm to approximately 30 mm and a height (H) of approximately 5 mm to approximately 15 mm.

8. The needle cover of claim 1, wherein the disinfecting pad is impregnated with an alcohol.

9. The needle cover of claim 1, wherein the disinfecting pad is impregnated with approximately 70% isopropyl alcohol.

10. The needle cover of claim 1, further comprising a mount coupled to the needle shield, wherein the disinfecting pad is coupled to the mount.

11. The needle cover of claim 1, wherein the disinfecting pad is coupled to the mount with an adhesive.

12. The needle cover of claim 11, wherein the mount is removably coupleable to the needle shield.

13. The needle cover of claim 11, wherein the mount is integrated with the needle shield such that they are a single, monolithic structure.

14. The needle cover of claim 11, wherein the mount comprises a pad mount coupled to the disinfecting pad and a shield sleeve surrounding a distal end of the needle shield.

15. The needle cover of claim 1, further comprising a protective film coupled with the disinfecting pad.

16. A syringe comprising:
a barrel;
a plunger at least partially received in the barrel;
a needle hub coupled to the barrel;
a needle coupled to the needle hub; and
a needle cover removably coupled to the needle hub; the needle cover comprising:
a needle shield; and
a disinfecting pad coupled to the needle shield, wherein the disinfecting pad is fixedly coupled to the needle shield with an adhesive, and wherein the disinfecting pad is a porous material impregnated with a disinfectant.

17. The syringe of claim 16, wherein a proximal end of the needle cover is removably coupled to the needle hub.

18. The syringe of claim 16, wherein the disinfecting pad is impregnated with approximately 70% isopropyl alcohol.

19. The syringe of claim 16, further comprising a protective film coupled to the disinfecting pad.

20. The syringe of claim 19, further comprising a mount coupled to the needle shield, wherein the disinfecting pad is coupled to the mount.
